(19) United States
(12) Patent Application Publication
(10) Pub. No.: US 2025/0375525 A1
(43) Pub. Date: Dec. 11, 2025

(54) USE OF INDOCYANINE GREEN IN ANTIBACTERIAL OR BACTERICIDAL APPLICATION

(71) Applicant: Buddhist Tzu Chi Medical Foundation, Hualien (TW)

(72) Inventors: Jong-Kai Hsiao, New Taipei City (TW); Meng-Xian Lin, Yilan County (TW); Hsiang-Ching Tseng, New Taipei City (TW)

(21) Appl. No.: 18/954,506

(22) Filed: Nov. 20, 2024

(30) Foreign Application Priority Data
Jun. 5, 2024 (TW) 113120724

(51) Int. Cl. A61K 41/00; A61P 31/04

(52) U.S. Cl. CPC A61K 41/008; A61P 31/04

(57) ABSTRACT

Infection of a Gram-negative bacterium causes adverse effects on human health and biological experiments. Accordingly, a use of indocyanine green for resisting the Gram-negative bacterium or sterilizing the Gram-negative bacterium is provided. The indocyanine green can also be used with photodynamic therapy to bring about better antibacterial or bactericidal effects.

### CROSS REFERENCE TO RELATED APPLICATION

This application claims the benefit of TW application No. 113120724, filed on Jun. 5, 2024. The content of the application is incorporated herein by reference.

The present application hereby incorporates by reference the entire contents of the text file named “TZC-P0004-USA-Sequence Listing.xml” in XML format. The text file containing the Sequencing Listing of the present application was created on Jun. 27, 2024 and is 1,778,044 bytes in size.

### BACKGROUND OF THE INVENTION

### 1. Field of the Invention

The present disclosure relates to antibacterial or bactericidal technology, particularly to the use of indocyanine green for resisting or sterilizing a bacterium.

### 2. Description of the Prior Art

Infections pose a serious threat to human health. Studies indicate that in 2019, approximately 7.7 million people worldwide died from bacterial infections, accounting for 13.6% of the total deaths. Gram-negative bacteria commonly cause disease in humans. For example, mycoplasma may cause any degree of respiratory symptoms, in which mycoplasma pneumonia is one of the most common manifestations of the infection. Clinically, patients initially experience symptoms such as headache, fever, sore throat, and general weakness. Two to five days later, they develop cough and sputum, and then even white sticky sputum or purulent sputum with blood streaks. The above symptoms may last for three to four weeks and may occur with other symptoms such as chills, sore throat, earache, vomiting, conjunctivitis, and skin rash. Antibiotics mainly treat mycoplasma pneumonia to alleviate symptoms and slow down the condition, but they cannot change the infectivity of bacteria. Therefore, mycoplasma pneumonia is still infectious during or after treatment. The illness, especially cough, can last for weeks. In addition to causing pneumonia, mycoplasma can also cause genitourinary tract infections and pelvic inflammatory diseases. Moreover, mycoplasma contamination, which is common during biological experiments, often leads to experimental failure and is quite detrimental to research progress.

Although mycoplasma infection causes many problems, general antibiotics (such as penicillins or β-lactam antibiotics) cannot exert a bactericidal effect on mycoplasma that does not have a cell wall. Also, antibiotic treatment may cause drug resistance problems.

Therefore, there is an unmet need in the art for methods of effectively combating or killing Gram-negative bacteria without causing drug resistance problems.

### SUMMARY OF THE INVENTION

In view of the foregoing, the present disclosure provides a use of indocyanine green for resisting or sterilizing a bacterium which is a Gram-negative bacterium.

The present disclosure will no doubt become obvious to those of ordinary skill in the art after reading the following detailed description of the embodiments that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the distribution changes of mycoplasma in human embryonic kidney cells 293 (HEK293 cells) observed by DAPI staining before and after light treatment with indocyanine green (ICG) in accordance with at least one embodiment of the present disclosure. After HEK293 cells are treated with 50 μg/mL ICG and irradiated with near-infrared light excitation, the distribution of mycoplasma in each group was observed by DAPI staining (the red arrow indicates the expression position of mycoplasma). CTL: control group; −Laser: not excited by laser; +Laser: excited by laser.

FIG. 2 shows the level changes of mycoplasma DNA in HEK293 cells analyzed by RT-PCR before and after light treatment with ICG in accordance with at least one embodiment of the present disclosure. (A) After HEK293 cells are treated with 50 μg/mL ICG and irradiated with near-infrared light excitation, the expression level of mycoplasma DNA in each group was observed by RT-PCR and DNA electrophoresis. (B) Results of DNA electrophoresis were quantitated by the Image J software (p-value: *<0.05, **<0.01, and ***<0.001).

FIG. 3 shows the distribution changes of mycoplasma in fibrosarcoma HT1080-1B3 cells observed by DAPI staining before and after light treatment with ICG in accordance with at least one embodiment of the present disclosure. After HT1080-1B3 cells are treated with 50 μg/mL ICG and irradiated with near-infrared light excitation, the distribution of mycoplasma in each group was observed by DAPI staining (the red arrow indicates the expression position of mycoplasma). CTL: control group; −Laser: not excited by laser; +Laser: excited by laser.

FIG. 4 shows the level changes of mycoplasma DNA in HT1080-1B3 cells analyzed by RT-PCR before and after light treatment with ICG in accordance with at least one embodiment of the present disclosure. (A) After HEK293 cells are treated with 50 μg/mL ICG and irradiated with near-infrared light excitation, the expression level of mycoplasma DNA in each group was observed by RT-PCR and DNA electrophoresis. (B) Results of DNA electrophoresis were quantitated by the Image J software (p-value: *<0.05, **<0.01, and ***<0.001).

### DETAILED DESCRIPTION

The following embodiments are provided to illustrate the present disclosure in detail. A person having ordinary skills in the art can easily understand the advantages and effects of the present disclosure after reading the specification of the present disclosure, and also can implement or apply that in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

The proportional probabilities, positions, quantities, and other features shown in the drawings attached hereto are only used to illustrate the embodiments described herein and are for a person having ordinary skill in the art to read and understand this disclosure, rather than to limit the scope of the present disclosure. Therefore, without affecting the purpose and effects that can be achieved by the present disclosure, any changes, modifications, or adjustments to the above features should fall within the scope of the technical content disclosed herein.

As used herein, the articles “a”, “an”, and “the” are used herein to refer to one or more than one (i.e., to at least one) of the grammatical object of the article, including plural referents unless expressly and unequivocally limited to one referent. The term “or” is used interchangeably with the term “and/or” unless the context clearly indicates otherwise.

As used herein, the term “about” is used herein to mean within the typical ranges of tolerances in the art. For example, “about” can be understood as about 2 standard deviations from the mean. When “about” is present before a series of numbers or a range, it is understood that “about” can modify each of the numbers in the series or range. For example, the numerical value is meant to encompass variations of ±20%, ±10%, ±5%, ±1%, ±0.5%, or =0.1% from the numerical value.

As used herein, the numeral ranges are inclusive and combinable, any numeral value that falls within the numeral scope herein could be taken as a maximum or minimum value to derive the sub-ranges therefrom. For example, it should be understood that the numeral range “700 nm to 1400 nm” comprises any sub-ranges between the minimum value of 700 nm to the maximum value of 1400 nm, such as the sub-ranges from 700 nm to 900 nm, from 800 nm to 1400 nm, and from 800 nm to 900 nm. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. In addition, a plurality of numeral values used herein can be optionally selected as maximum and minimum values to derive numerical ranges; for instance, the numerical ranges of 700 nm to 800 nm, 700 nm to 900 nm, or 800 nm to 900 nm can be derived from the numeral values of 700 nm, 800 nm, and 900 nm.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “effective amount” refers to a sufficient amount of an active ingredient to provide an antibacterial or bactericidal effect with a reasonable beneficial-harmful ratio. Within the scope of appropriate medical judgment, the amount and proportion of the active ingredient will depend upon the condition of the affected area, the extent of the symptoms, the cause of the disease, the duration of treatment, the specific active ingredient present, the concentration, the method of administration, or the patient's overall condition. These may vary depending on factors such as the condition, the individual's tolerance to the administration, and other drugs being administered to the individual.

As used herein, the term “administering” or “administration” refers to the placement of an active ingredient into a subject part or cell by a method or route that results in at least partial localization of the active ingredient at a desired site to produce the desired effect. In some embodiments, the active ingredient described herein can be administered by any suitable route known in the art, such as, but not limited to, topical administration to the skin of a subject or addition to a cell culture environment.

As used herein, the term “subject” refers to a human or other animal. Examples of the subject include, but are not limited to, human, monkey, mouse, rat, woodchuck, ferret, rabbit, hamster, cow, horse, pig, deer, dog, cat, fox, wolf, chicken, emu, and ostrich. In at least one embodiment of the present disclosure, the subject is a mammal, e.g., a primate such as a human.

As described in this article, the term “photodynamic therapy” refers to photochemical therapy, which is based on the induction of oxygen by photosensitizers to produce reactive oxygen species, which have a killing effect on tumor cells or pathogenic microorganisms to achieve the purpose of treating diseases.

As used herein, “HT1080-1B3 cells” refer to fibrosarcoma cells transfected with the SLCO1B3 gene. Compared to human embryonic kidney cells HEK293, HT1080-1B3 cells consistently overexpress the OATP1B3 transport protein (transcribed from the SLCO1B3 gene), and ICG is the substrate of the OATP1B3 transport protein. Therefore, compared with HEK293 cells, HT1080-1B3 cells can ingest more ICG.

As used herein, the term “Gram-negative bacterium” refers to bacteria that do not retain crystal violet dye in Gram staining protocols, including but not limited to: Enterobacteriaceae, such as but not limited to: Escherichia (including E. coli), Klebsiella, Enterobacter, Citrobacter, Salmonella or Shigella; Yersiniaceae, such as but not limited to: Serratia; Morganellaceae, such as but not limited to: Proteus or Providencia, Pseudomonadaceae, such as but not limited to: Pseudomonas (including Pseudomonas aeruginosa); Moraxellaceae, such as but not limited to: Moraxella (including M.catarrhalis); Pasteurellaceae, such as but not limited to: Haemophilus; Neisseriaceae, such as but not limited to: Neisseria; Mycoplasmataceae, such as but not limited to: Mycoplasma.

As used herein, the term “mycoplasma” is the smallest prokaryotic organism between independent life and intracellular parasitic life, and has no cell wall structure. Its structure is similar to that of typical bacteria and belongs to the Bacteria domain. It has no cell wall, and thus presents as Gram-negative. Its shape is easy to change and is sensitive to osmotic pressure, but is not sensitive to antibiotics that inhibit cell wall synthesis. In the present disclosure, the abbreviation M. preceding the species name represents Mesomycoplasma or Mycoplasma.

As used herein, the term “bacterial infection” refers to infection by bacteria and includes systemic infections (bacteremia and sepsis) and/or infection of any organ or tissue of the body. Such organs or tissues include, but are not limited to, skeletal muscle, skin, bloodstream, kidneys, heart, lungs, and bones. Such infections can be caused by Gram-negative bacteria as described herein.

As described herein, the term “sterilizing,” or “bactericidal” means the killing of microorganisms; the term “antibacterial” means the effect of inhibiting and slowing down the growth and proliferation of microorganisms, the effect of preservatives, and/or the effect of extending the validity period. In some embodiments, the microorganisms include, but are not limited to, bacteria, viruses, mold, algae, and/or fungi. In some embodiments, “sterilizing,” “bactericidal,” or “antibacterial” effect may be directed against microorganisms in materials, surfaces, environments, culture media, or individuals. In some embodiments, “sterilizing,” “bactericidal,” or “antibacterial” may be directed to specific one or more microorganisms, but not all microorganisms.

In at least one embodiment of the present disclosure, the use of indocyanine green for resisting or sterilizing a bacterium may include irradiating the indocyanine green and/or the Gram-negative bacteria with near-infrared light.

In at least one embodiment of the present disclosure, the near-infrared light may have a wavelength of about 700 nm to about 1400 nm, such as but not limited to: about 750 nm to about 1100 nm, about 800 nm to about 1000 nm, or about 850 nm to about 900 nm. In some embodiments, the near-infrared light may have a wavelength of about 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 1000 nm, 1100 nm, 1200 nm, 1300 nm, 1310 nm, 1320 nm, 1330 nm, 1340 nm, 1350 nm, 1360 nm, 1370 nm, 1380 nm, or 1390 nm, but the present disclosure is not limited thereto.

In at least one embodiment of the present disclosure, the indocyanine green may emit about 800 nm to about 850 nm (for example, but not limited to: about 805 nm, 810 nm, 820 nm, 825 nm, 830 nm, 835 nm, 840 nm, or 845 nm) of red fluorescence, after being excited by near-infrared light with a wavelengths from about 700 nm to about 1400 nm.

In at least one embodiment of the present disclosure, the Gram-negative bacterium may be free of a cell wall.

In at least one embodiment of the present disclosure, the Gram-negative bacterium may be a Mycoplasmatota.

In at least one embodiment of the present disclosure, the Gram-negative bacterium may be a Mollicutes.

In at least one embodiment of the present disclosure, the Gram-negative bacterium may be a Mycoplasmatales.

In at least one embodiment of the present disclosure, the Gram-negative bacterium may be a Mycoplasmataceae.

In at least one embodiment of the present disclosure, the Gram-negative bacterium may be a Mycoplasma.

In at least one embodiment of the present disclosure, the mycoplasma may be M. genitalium, a M. fermentans, a M. salivarium, a M. hominis, a M. pneumonia, a M. incognitus, a M. penetrans, a M. pirum, a M. faucium, a M. lipophilum, a M. buccale, a M. cloacale, a M. gallinarum, a M. gallisepticum, a M. gallopavonis, a M. glycophilum, a M. iners, a M. iowae, a M. lipofaciens, aM. meleagridis, a M. synoviae, a M. flocculare, a M. hyopneumoniae, a M. hyorhinis, a M. hyosynoviae, a M. capricolum subsp. capricolum, a M. capricolum subsp. capripneumoniae, a M. mycoides subsp. mycoides LC, a M. mycoides subsp. capri, a M. bovis, a M. bovoculi, a M. canis, a M. californicum, a M. dispar, a mycoplasma unnamed species 1654_15 chromosome, or any combination thereof.

In some embodiments, the mycoplasma may be a M. hyorhinis or a mycoplasma unnamed species 1654_15 chromosome.

In at least one embodiment of the present disclosure, the genome of mycoplasma is about 0.48 to about 1.38 million bases (for example, but not limited to: 0.5, 0.55, 0.58, 0.59, 0.6, 0.65, 0.70, 0.75, 0.80, 0.84, 0.85, 0.90, 0.91, 0.92, 0.93, 0.94, 0.95, 1.0, 1.05, 1.1, 1.15, 1.2, 1.25, 1.3, or 1.35 million bases).

In at least one embodiment of the present disclosure, the use of indocyanine green for resisting or sterilizing a bacterium may include administering to a subject in need thereof or a cell in need thereof an effective amount of the indocyanine green. In at least one embodiment of the present disclosure, the use of indocyanine green for resisting or sterilizing a bacterium may include administering to a subject in need thereof or a cell in need thereof an effective amount of the indocyanine green and irradiating the indocyanine green with near-infrared light excitation. In some embodiments, the effective amount may be about 5 μg/mL to about 100 μg/mL, such as but not limited to: about 10 μg/mL to about 90 μg/mL, about 20 μg/mL to about 80 μg/mL, or about 40 μg/mL to about 70 μg/mL. In some embodiments, the effective amount may be about 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, or 100 μg/mL.

In at least one embodiment of the present disclosure, the indocyanine green enters the cell with the assistance of the OATP1B3 transport protein.

In at least one embodiment of the present disclosure, the use of indocyanine green for resisting or sterilizing a bacterium may include photodynamic therapy. In some embodiments, compared to other bacteria, the indocyanine green has a better antibacterial or bactericidal effect against the Gram-negative bacterium. For example, the indocyanine green can reduce the distribution of mycoplasma and the level expression of mycoplasma DNA after exciting irradiation with near-infrared light without the need for any antibiotic assistance, and thus can provide a novel therapeutic strategy for the clinical treatment of infections.

In at least one embodiment of the present disclosure, the use of indocyanine green for resisting or sterilizing a bacterium may also be administered in combination with antibiotics to maintain and enhance the antibacterial or bactericidal effect of photodynamic therapy. In some embodiments, the dosage of antibiotics may be reduced due to the excellent antibacterial or bactericidal effect produced by photodynamic therapy.

### EXAMPLE

Exemplary embodiments of the present disclosure are further described in the following examples, which should not be construed to limit the scope of the present disclosure.

### Materials and Methods

Indocyanine green, also known as Diagnogreen or indocyanine green (ICG), was purchased from Daiichi Sankyo Taiwan Ltd. Fibrosarcoma cells HT1080-1B3 and human embryonic kidney cells HEK293 transfected with SLCO1B3 gene were obtained from the laboratory of Dr. HSIAO, JONG-KAI of Buddhist Foundation Taipei Tzu Chi Hospital. The cell culture materials include cell culture medium DMEM and DMEM-high glucose, fetal bovine serum (FBS), penicillin-streptomycin, and 0.05% trypsin-EDTA, and all the materials were purchased from GIBCO of the United States. 4′,6-diamidino-2-phenylindole (DAPI) dye and KAPA rapid extraction kit were purchased from Sigma-Aldrich, and e-Myco™ VALID Mycoplasma PCR detection kit was purchased from iNtRON Biotechnology.

### Cell Culture

HT1080-1B3 cells were cultured in a DMEM-high glucose culture medium containing 10% FBS, mycoplasma, and 1% penicillin-streptomycin. HEK293 cells were cultured in a DMEM medium containing 10% FBS and 1% penicillin-streptomycin. When cell subculture was performed, the cells were firstly washed with phosphate buffer solution (PBS) and reacted with 0.05% trypsin-EDTA at 37° C. for 5 minutes to remove cells from the culture dish. Then, the culture medium was used to terminate the trypsin-EDTA reaction, and centrifugation (revolutions per minute was 1200 rpm, and time was maintained for 5 minutes) was performed. Finally, the supernatant was removed and fresh culture medium was added to reconstitute cell pellets, and then reseeded on various culture dishes according to experimental needs.

### DAPI Staining

First, HT1080-1B3 and HEK293 cells were collected through trypsin-EDTA reaction and then seeded into a six-well plate at a cell number of 3×10⁵ cells/well. The next day, a working concentration of 50 μg/mL of ICG was added to each well, and the reaction took place in the dark at 37° C. for 1 hour. After the reaction was completed, the cells were irradiated with near-infrared light of 780 nm wavelength for 10 minutes. The supernatant was removed, and the cells were washed twice with PBS. Then, a working concentration of 1 μg/ml DAPI was added to each well, and the reaction took place in the dark at room temperature for 15 minutes. Finally, washed twice with PBS, and the quantitative changes of stained mycoplasma chromosomes were observed under the UV light mode of an inverted fluorescence microscope (Nikon).

### RT-PCR

HT1080-1B3 and HEK293 cells were collected through trypsin-EDTA reaction and then seeded into a six-well plate at a cell number of 3×10⁵ cells/well. The next day, a working concentration of 50 μg/mL of ICG was added to each well, and the reaction took place in the dark at 37° C. for 1 hour. After the reaction was completed, the cells were irradiated with near-infrared light of 780 nm wavelength for 10 minutes, the supernatant was removed, and the cells were washed twice with PBS. Then, the cells were collected into a 1.5 mL test tube with trypsin-EDTA, 100 μL KAPA rapid extraction buffer was added to the test tube, and the test tube was placed in a dry bath at 75° C. for 10 minutes and at 95° C. for 5 minutes. Then, DNA samples were prepared according to the operating manual of the e-Myco™ VALID Mycoplasma PCR detection kit, and PCR reaction was performed by PCR instrument (Applied Biosystems) under setting conditions (30 cycles: 95° C./3 minutes, 95° C./15 seconds, 55° C./15 seconds, 72° C./20 seconds and 72° C./1 minute). Finally, DNA electrophoresis (2% agarose, 100 V, 40 minutes) was performed and observed by the electrophoresis gel UV image capture system (Clubio).

### RNA-Seq Materials and Methods

Regarding RNA preparation and quality control, total RNA was prepared by silica gel column chromatography. The RNA sample was connected to the silica gel column, treated with DNase I, and washed thoroughly before elution. ND-1000 spectrophotometer and Agilent 2100 Bioanalyzer were used, and RNA 6000 Nano assay was used to determine RIN value (the RIN value is the standard of measurement of RNA degradation quality) based on the above steps to detect quality and the quantity of purified RNA.

rRNA-dep RNA library construction and sequencing: aliquots of 1,000 ng RNA were input into Illumina TruSeq standard total RNA and Ribo-Zero library preparation workflow according to the manufacturer's operating manual (Illumina, Cat #20020598). The quality and quantity of the rRNA-dep RNA library were confirmed by the Agilent TapeStation 4200 system with D1000 ScreenTape detection. Multiple RNA libraries were mixed, and the multiple RNA libraries were performed sequencing by Novaseq 6000 sequencer with 2×150 paired-end sequencing protocol. Sequencing data (FASTQ format) was generated using Illumina's base calling program bcl2fastq. Adapter and quality splicing were performed via Trimmomatics.

Data analysis: sequencing data was compared with the GRCh38 human reference genome via HISAT2 aligner, and mycoplasma sequences were obtained by comparative analysis of the genomes of species whose sequences cannot be compared with the human genome. Differential performance analysis was performed using StringTie and DEseq with genomic bias detection/correction and Welgene Biotech's in-house production line. Functional enrichment test of genes with different expressions in individual experimental designs is performed using clusterProfiler.

### Example 1

After administering ICG and near-infrared light (780 nm) excitation and irradiation to HEK293 cells and HT-1080-1B3 cells, observing the cell survival rates of different concentration groups by MTT analysis. The results show that ICG and near-infrared light excitation and irradiation have no negative impact on cells (not shown in Drawings).

### Example 2

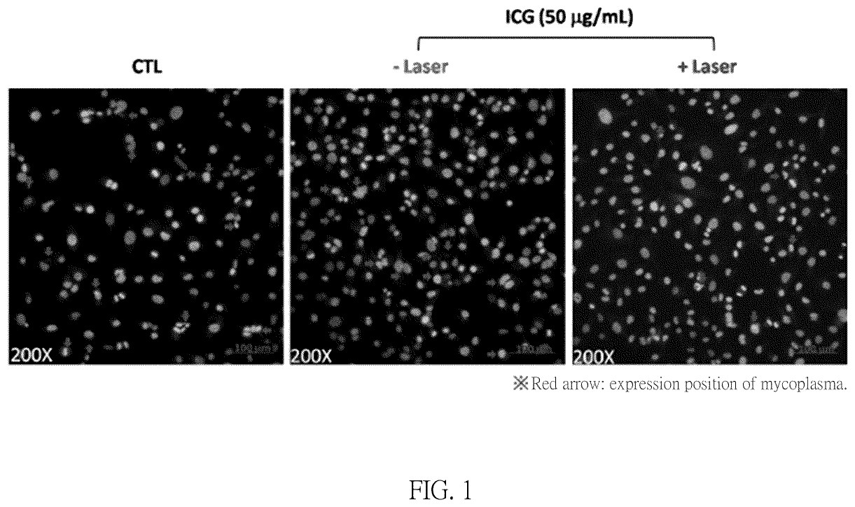

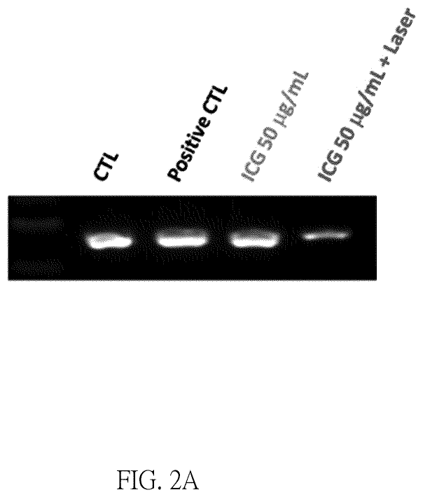

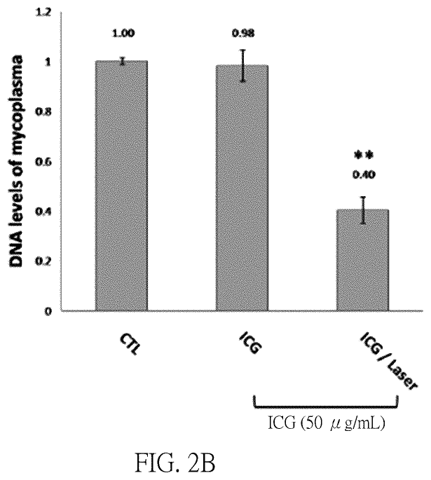

As shown in FIG. 1, it is observed by DAPI staining that the number of mycoplasma bacteria (red arrow) is significantly reduced after administration of ICG and near-infrared light excitation irradiation (50 μg/mL ICG+780 nm laser) to HEK293 cells. As shown in FIG. 2, administration of ICG and near-infrared light excitation irradiation (50 μg/mL ICG+780 nm laser) to HEK293 cells can significantly reduce the expression level of mycoplasma DNA.

### Example 3

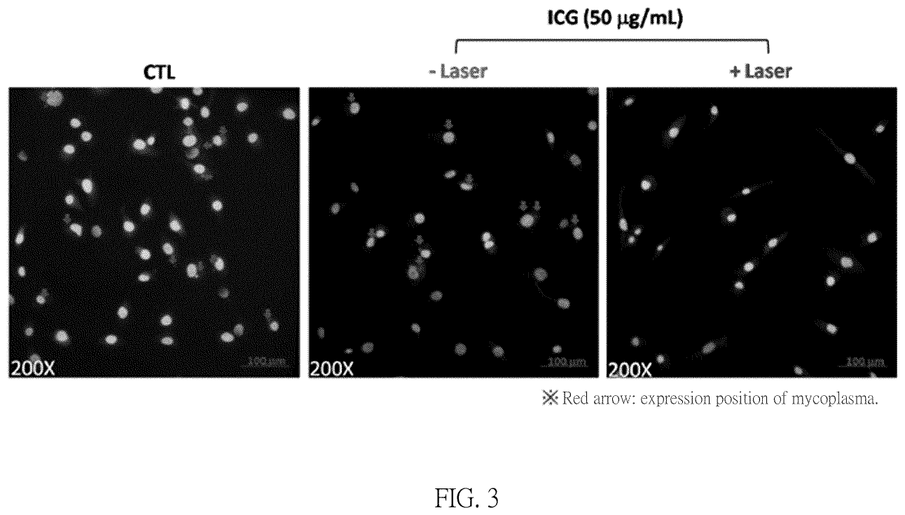

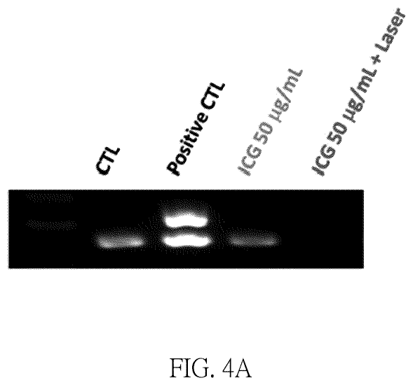

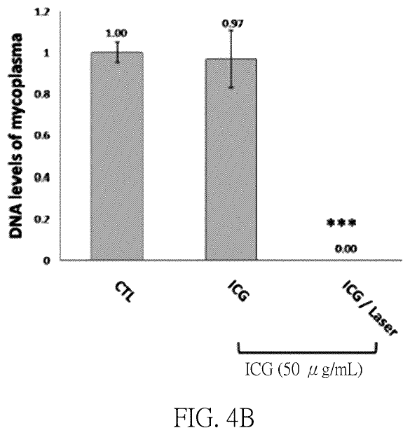

As shown in FIG. 3, it is observed by DAPI staining that the number of mycoplasma bacteria (red arrow) disappears after administration of ICG and near-infrared light excitation irradiation (50 μg/mL indocyanine green+780 nm laser) to HT-1080-1B3 cells. As shown in FIG. 4, after administration of ICG and near-infrared light excitation irradiation (50 μg/mL indocyanine green+780 nm laser) to HT-1080-1B3 cells, no expression level of mycoplasma DNA was observed.

It can be seen from the results of Example 2 and Example 3 that ICG and near-infrared light excitation and irradiation exhibit excellent antibacterial or bactericidal effects on different cell lines. However, the effects on HT1080-1B3 cells is better than that in HEK293 cells since HT1080-1B3 cells, which continue to overexpress the OATP1B3 transport protein (ICG is the substrate of the OATP1B3 transport protein), can uptake more ICG than HEK293 cells. Accordingly, combination of ICG and photodynamic therapy is more effective on HT1080-1B3 cells than HEK293 cells. These results also prove that ICG can directly affect mycoplasma.

### Example 4

Sequencing the mycoplasma of the above Examples shows CP035041.1 Mesomycoplasma hyorhinis strain JF5820 chromosome (complete genome, SEQ ID NO. 1) and CP051214.1 Mycoplasma sp. 1654_15 chromosome (complete genome, SEQ ID NO. 2).

Those skilled in the art will readily observe that numerous modifications and alterations of the method and use may be made while retaining the teachings of the invention. Accordingly, the above disclosure should be construed as limited only by the metes and bounds of the appended claims.

What is claimed is:

1. A use of indocyanine green for resisting or sterilizing a bacterium, wherein the bacterium is a Gram-negative bacterium.

2. The use of claim 1, comprising irradiating the indocyanine green and/or the Gram-negative bacteria with near-infrared light.

3. The use of claim 2, wherein the near-infrared light has a wavelength of about 700 nm to about 1400 nm.

4. The use of claim 1, wherein the Gram-negative bacterium is free of a cell wall.

5. The use of claim 1, wherein the Gram-negative bacterium is a Mycoplasmatota.

6. The use of claim 5, wherein the Gram-negative bacterium is a Mollicutes.

7. The use of claim 6, wherein the Gram-negative bacterium is a Mycoplasmatales.

8. The use of claim 7, wherein the Gram-negative bacterium is a Mycoplasmataceae.

9. The use of claim 8, wherein the Gram-negative bacterium is a Mycoplasma.

10. The use of claim 1, comprising administering to a subject in need thereof or a cell in need thereof an effective amount of the indocyanine green.

11. The use of claim 10, wherein the effective amount is 5 μg/mL to 100 μg/mL.

12. The use of claim 10, wherein the indocyanine green enters the cell by assistance of OATP1B3 transport protein.

13. The use of claim 10, wherein the subject or the cell does not treated with an antibiotic.

14. The use of claim 10, further comprising administering an antibiotic to the subject or the cell.
